(19) United States
(12) Patent Application Publication
(10) Pub. No.: US 2017/0065395 A1
(43) Pub. Date: Mar. 9, 2017

(54) INJECTABLE VALVE AND OTHER FLOW CONTROL ELEMENTS

(71) Applicant: AMSEL MEDICAL CORPORATION, Cambridge, MA (US)

(72) Inventors: Raanan A. Miller, Cambridge, MA (US); Arnold Miller, Cambridge, MA (US)

(21) Appl. No.: 15/350,872

(22) Filed: Nov. 14, 2016

Related U.S. Application Data
(63) Continuation of application No. 13/888,165, filed on May 6, 2013.
Application No. 13/888,165 is a continuation-in-part of application No. 13/857,424, filed on Apr. 5, 2013.
Application No. 13/857,424 is a continuation-in-part of application No. 13/348,416, filed on Jan. 11, 2012.
(60) Provisional application No. 61/620,787, filed on Apr. 5, 2012.
(60) Provisional application No. 61/431,609, filed on Jan. 11, 2011.
(60) Provisional application No. 61/643,092, filed on May 4, 2012.

(51) Int. Cl. A61F 2/01; A61B 17/128; A61F 2/24; A61B 17/12

(52) U.S. Cl. CPC A61F 2/01; A61B 17/12022; A61B 17/12136; A61B 17/1285; A61F 2/2475; A61B 2017/00867

(57) ABSTRACT

Flow control elements can be injectably delivered through a needle (e.g., a fine hypodermic needle) or other tube and deployed into a blood vessel, percutaneously or laparoscopically, to effect the directional blood flow within the vessel. The valve (or other blood flow or occlusion control element) is delivered from outside the blood vessel directly to the chosen site within the blood vessel. The flow control elements are minimally invasive, reducing pain, discomfort and risk to the patient, while reducing the time the procedure takes to perform. In addition, the accuracy of deployment of the valve or other flow control element can be very precisely controlled.

### REFERENCE TO PENDING PRIOR PATENT APPLICATION

This patent application:
(i) is a continuation-in-part of pending prior U.S. patent application Ser. No. 13/857,424, filed Apr. 5, 2013 by Arnold Miller et al. for METHOD AND APPARATUS FOR OCCLUDING A BLOOD VESSEL (Attorney's Docket No. AM-9), which patent application is (a) a continuation-in-part of prior U.S. patent application Ser. No. 13/348,416, filed Jan. 11, 2012 by Arnold Miller et al. for METHOD AND APPARATUS FOR TREATING VARICOSE VEINS (Attorney's Docket No. AM-0708), which patent application claims benefit of prior U.S. Provisional patent application Ser. No. 61/431,609, filed Jan. 11, 2011 by Arnold Miller for METHOD AND APPARATUS FOR TREATING VARICOSE VEINS (Attorney's Docket No. AM-7 PROV), and (b) claims benefit of prior U.S. Provisional Patent Application Ser. No. 61/620,787, filed Apr. 5, 2012 by Arnold Miller et al. for TEMPORARY ARTERIAL OCCLUSION FOR MILITARY AND CIVILIAN EXTREMITY TRAUMA (Attorney's Docket No. AM-9 PROV); and
(ii) claims benefit of pending prior U.S. Provisional Patent Application Ser. No. 61/643,092, filed May 4, 2012 by Raanan A. Miller et al. for INJECTABLE VALVE AND OTHER FLOW CONTROL ELEMENTS (Attorney's Docket No. AM-10 PROV), which patent application is hereby incorporated herein by reference.

The five (5) above-identified patent applications are hereby incorporated herein by reference.

### FIELD OF THE INVENTION

This invention relates to surgical methods and apparatus in general, and more particularly to surgical methods and apparatus for treating blood vessels and other tubular structures.

### BACKGROUND OF THE INVENTION

Chronic venous disease is an extremely common disease in the general population. The most common cause of the disease is the development of venous valvular incompetence in the deep veins of the lower extremities. Not all of the causes of this valvular incompetence are known, but the development of clots within the veins (i.e., deep vein thrombosis) is one of the most common recognizable causes of valvular incompetence. The causes of deep vein thrombosis may be local within the veins themselves, such as trauma to a particular vein or slowing of the blood flow in these veins from lack or mobility (e.g., such as occurs during a long airplane flight), or systemic problems related to the blood coagulation, etc.

These clots organize around the valves in the vein which are necessary for directing the flow of blood from the extremities to the heart, and destroy these valves. Over time (e.g., months to years), the clot obstructing the veins may recanalize, thereby allowing renewed blood flow. Venous flow in the lower extremities is controlled by muscular contraction. The combination of obstruction of the veins of the lower extremities, and valvular incompetence, results in the generation of high venous pressures within the lower extremities which, over time, results in the condition of chronic venous disease with swelling, pigmentation, ulceration, infection and progressive disability.

The closer the incompetent valve is to the heart, the more severe the clinical consequences. Currently, treatment measures are aimed at alleviating the consequences of the high venous pressures, e.g., with leg elevation and/or compression stockings.

Because of technical challenges, attempts to replace (or repair) the incompetent venous valves surgically and by utilizing catheter-directed techniques remain in the research domain and have not yet achieved clinical acceptance.

### SUMMARY OF THE INVENTION

In one embodiment of the present invention, the clinical need is addressed with a device that provides a simplified method of placing a competent valve through a needle (e.g., a fine hypodermic needle) or other tube to within any incompetent vein, as diagnosed with modern imaging techniques, e.g., ultrasound or fluoroscopy. While replacement valves delivered through a blood vessel are potentially most useful in the venous system, they can also be deployed throughout the vascular and organ systems of the body.

The present invention relates to various flow control elements that can be injectably delivered through a needle (e.g., a fine hypodermic needle) or other tube and deployed into a blood vessel, percutaneously or laparoscopically, whereby to effect the directional blood flow within the vessel. The valve (or other blood flow or occlusion control element) is delivered from outside the blood vessel directly to the chosen site within the blood vessel.

The present invention is designed to be minimally invasive, reducing pain, discomfort and risk to the patient, while reducing the time the procedure takes to perform. In addition, the accuracy of deployment of the valve or other flow control element can be very precisely controlled. The procedure is performed with external imaging including, but not limited to, ultrasound, fluoroscopy and/or other visualization methods. The present invention does not require the use of tumescent anesthetic.

In one preferred form of the invention, an injectable valve is positioned within the blood vessel. Once deployed within the blood vessel, the valve allows the blood to flow in one direction only, restricting blood flow in the reverse direction. Unique aspects of this valve include the way the valve is delivered and inserted into a blood vessel.

In other preferred forms of the invention, other flow control elements may be injected within the blood vessel, e.g., a filter, an occluder, a balloon, a polymer occluder, a transvascular screw, a transvascular clamp, etc.

In another preferred form of the invention, the injectable valve (or other flow control device) may be percutaneously or laparoscopically delivered to an artery through a needle (e.g., a fine hypodermic needle) or other tube, e.g., so as to replace a defective aortic valve.

In yet another preferred form of the invention, the injectable valve (or other flow control device) may be cleared of thrombus periodically (or as needed) via an anti-coagulant coating, or via a thrombolytic agent deposited on the flow control device, or via the delivery of an anticoagulant compound or thrombolytic agent which is delivered externally to the vessel, but which flows through a channel or channels in the support frame of the flow control device and is delivered proximate to the flow control device. It should be appreciated that the anti-coagulant compound or thrombolytic agent may be replaced by other drugs for treating specific conditions associated with the vessel, organ or patient, e.g., to reduce pain or inflammation, or to deliver chemotherapeutic drugs, etc.

In one preferred form of the invention, there is provided apparatus for controlling flow through a body lumen, the apparatus comprising:
an injectable flow control device for disposition within the body lumen, the injectable flow control device comprising:
   a resilient frame for seating against the inside wall of the body lumen and compressible for disposition within a tube for delivery through the side wall of the body lumen to the interior of the body lumen, the resilient frame having an opening therein; and
   a flow restrictor in contact with the resilient frame for restricting flow through the opening of the resilient frame.

In another preferred form of the invention, there is provided apparatus for controlling flow through a body lumen, the apparatus comprising:
an injectable flow control device for disposition within the body lumen, the injectable flow control device comprising:
   a balloon for seating against the inside wall of the body lumen and compressible for disposition within a tube for delivery through the side wall of the body lumen to the interior of the body lumen.

In another preferred form of the invention, there is provided apparatus for controlling flow through a body lumen, the apparatus comprising:
an injectable flow control device for disposition within the body lumen, the injectable flow control device comprising:
   a flowable material for solidifying against the inside wall of the body lumen, wherein at least one portion of the solidified material extends through the side wall of the body lumen, whereby to secure the solidified material in the body lumen.

In another preferred form of the invention, there is provided a method for controlling flow through a body lumen, the method comprising:
advancing a tube through the side wall of a body lumen; and
positioning an injectable flow control device within the body lumen, the injectable flow control device controlling flow through a body lumen.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1 and 2 are schematic views showing a novel valve formed in accordance with the present invention;

FIGS. 3 and 4 are schematic views showing another novel valve formed in accordance with the present invention;

FIG. 5 is a schematic view showing the novel valve of FIGS. 1 and 2 disposed across a blood vessel;

FIG. 6 is a schematic view showing another form of novel valve disposed across a blood vessel;

FIGS. 7-15 are schematic views showing the novel valve of FIGS. 1 and 2 being deployed across a blood vessel;

FIG. 16 is a schematic view showing another form of novel valve formed in accordance with the present invention;

FIG. 17 is a schematic view showing another form of novel valve formed in accordance with the present invention;

FIG. 18 is a schematic view showing a novel filter formed in accordance with the present invention;

FIGS. 19 and 20 are schematic views showing a novel occluder formed in accordance with the present invention;

FIGS. 21-23 are schematic views showing a flow control device disposed across a blood vessel which can deliver fluidic compounds around the flow control device, whereby to prevent thrombus build-up around the flow control device;

FIGS. 24 and 25 are schematic views showing another novel valve or occluder formed in accordance with the present invention;

FIGS. 26 and 27 are schematic views showing the novel valve or occluder of FIGS. 24 and 25 being deployed within a blood vessel;

FIGS. 28-30 are schematic views showing a novel balloon formed in accordance with the present invention;

FIGS. 31-33 are schematic views showing a novel polymer occluder formed in accordance with the present invention;

FIGS. 34-36 are schematic views showing a novel transvascular screw formed in accordance with the present invention; and

FIGS. 37-43 are schematic views showing a novel transvascular clamp formed in accordance with the present invention.

### DETAILED DESCRIPTION OF THE PREFERRED EMBODIMENTS

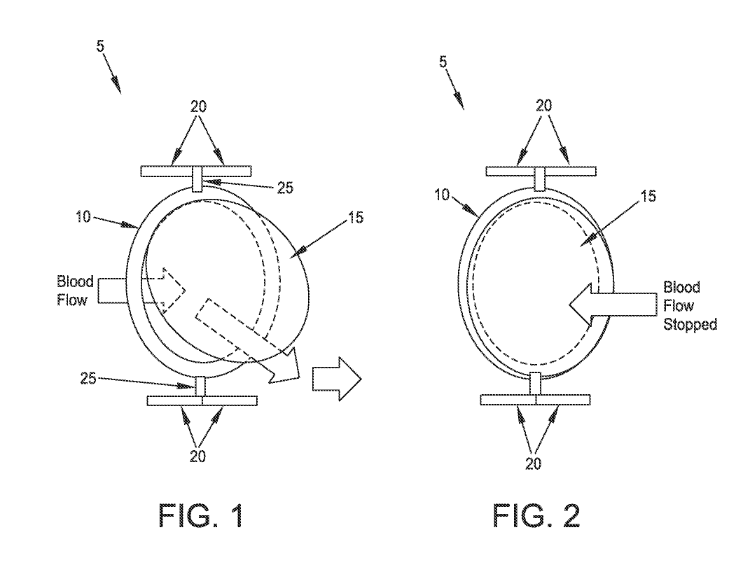

FIGS. 1 and 2 show a novel flow control device formed in accordance with the present invention. More particularly, FIGS. 1 and 2 show a novel valve 5 which may be used to control flow in a blood vessel or other body lumen. FIG. 1 shows valve 5 in an open configuration. FIG. 2 shows valve 5 in a closed configuration. In a preferred embodiment of the present invention, valve 5 comprises a resilient frame 10 having an attached resilient flap 15. Frame 10 and flap 15 have a generally circular configuration to match the cross-sectional geometry of a blood vessel. Frame 10 is designed such that it acts as a stop for flap 15, thereby limiting movement of flap 15 in one direction. Frame 10 may be made of a thicker material than flap 15, or of a different material than flap 15, or the same material and/or thickness as flap 15, etc. Frame 10 is designed to be attached to a blood vessel in such a way as to restrict, at least in part, movement of frame 10 vis-à-vis the blood vessel. Flap 15 may be attached to frame 10 via a hinge, solder, welding, etc., or flap 15 may be a contiguous part of frame 10 (i.e., flap 15 may be formed integral with frame 10). Flap 15 may comprise one or more movable components.

FIG. 1 shows valve 5 in an open-valve configuration, allowing blood flow through valve 5. FIG. 2 shows valve 5 in a closed-valve configuration, whereby blood flow is impeded.

Flap 15 and frame 10 may be formed by laser cutting, embossing, injection molding, or any other method known to those skilled in the art. In one preferred form of the invention, frame 10 also includes a plurality of resilient finger anchors 20 for attaching frame 10 to the wall of a blood vessel by penetrating through the wall of the blood vessel. It should be appreciated that frame 10 may comprise other elements for anchoring frame 10 to the wall of a blood vessel instead of, or in addition to, finger anchors 20.

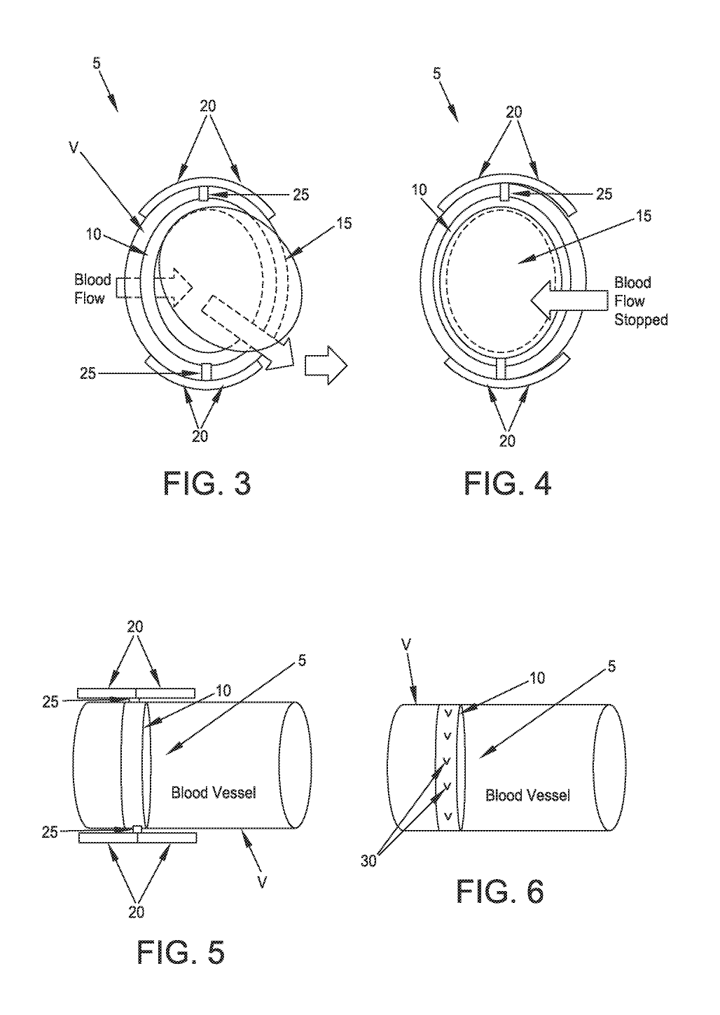

In another preferred form of the invention, and looking now at FIGS. 3 and 4, valve 5 can include flexible finger anchors 20, for example of Nitinol, which contact, or even conform to, the shape of the external wall of the blood vessel V, sandwiching the blood vessel V at least in part on either side, and exerting a force between finger anchors 20 and frame 10. This embodiment of the invention enables the finger anchors to better support frame 10, and distribute the pressure of the stopped blood across blood vessel V, thus preventing the valve 5 from moving or migrating within blood vessel V.

Looking next at FIGS. 5 and 6, valve 5 is shown deployed inside a blood vessel V. In a preferred form of the present invention, valve 5 may be deployed with finger anchors 20 spanning the wall of blood vessel V and deploying external to blood vessel V, whereby to compress against the exterior of blood vessel V and secure valve 5 in position. See FIG. 5. A connector 25, which may be formed as part of frame 10, connects frame 10 (disposed inside of blood vessel V) and finger anchors 20 (disposed outside of blood vessel V), with connector 25 penetrating the wall of blood vessel V.

In another preferred form of the present invention, and looking now at FIG. 6, there is shown a valve 5 which does not have finger anchors 20 disposed external to blood vessel V. In this form of the present invention, frame 10 includes anchors (or protruding elements) 30 disposed along the outer perimeter of frame 10, which secure frame 10 of valve 5 to the internal wall of blood vessel V, thereby anchoring frame 10 (and hence valve 5) to the wall of blood vessel V. Compression of valve 5 against the internal wall of blood vessel V may also be sufficient to secure valve 5 within blood vessel V, in which case anchors 30 may be omitted.

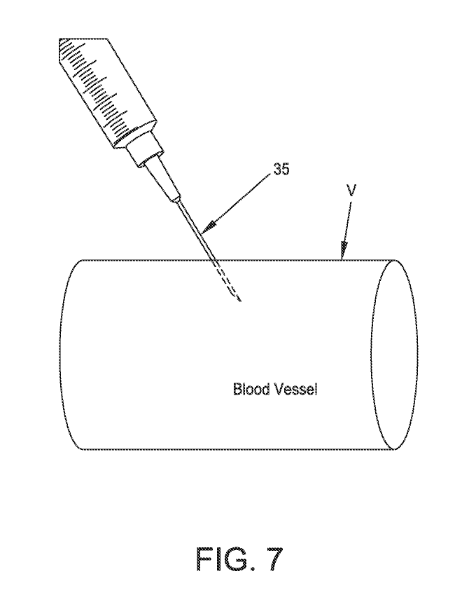

A novel feature of the present invention is the minimally invasive manner in which valve 5 is deployed within a blood vessel. The minimally invasive approach utilized by the present invention minimizes discomfort and trauma to the patient, and minimizes the risk of complications associated with surgical exposures. In one preferred form of the present invention, an anesthetic is delivered superficially and locally on the skin of the patient at the site of delivery of valve 5. More particularly, and looking now at FIGS. 7-9, valve 5 may be percutaneously deployed into a blood vessel V using a hollow needle 35 or other tube (preferably sharpened so as to facilitate passage of the tube through the side wall of the blood vessel, as well as through any intervening tissue). Valve 5 is compressed (or rolled-up) so that it fits within the bore of hollow needle 35. The frame 10, as well as the finger anchors 20 (or anchors 30) of valve 5 are also compressed to fit into the needle. Alternatively, valve 5 may be compressed and contained within a sheath 40 which is inserted into the bore of hollow needle 35. Sheath 40 affords additional control over the deployment process.

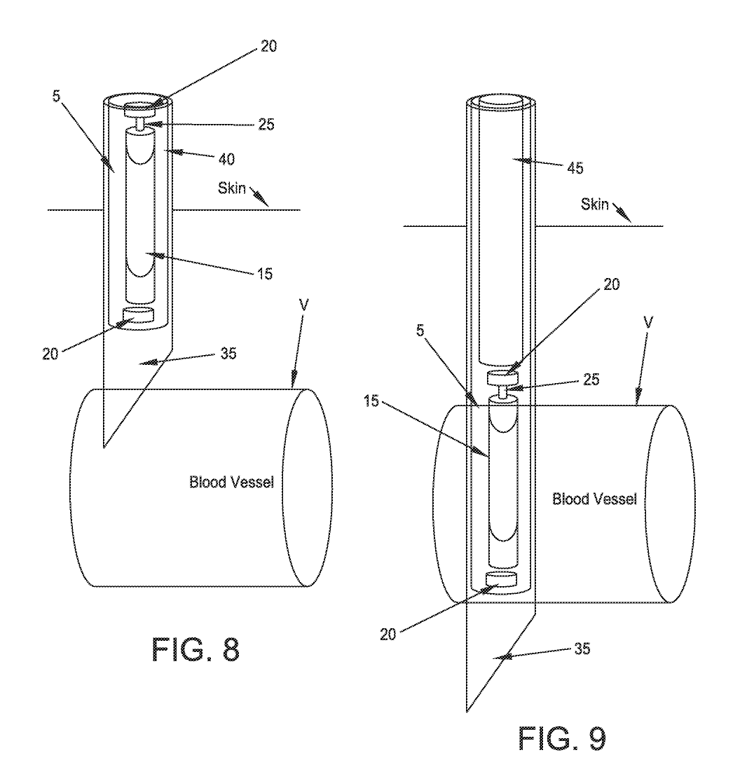

In one preferred form of the present invention, and as seen in FIGS. 8-14, frame 10 and flap 15 are rolled up along a single axis (e.g., in the manner of rolling a pancake on itself), whereby to facilitate positioning valve 5 within needle 35, i.e., by aligning the longitudinal axis of the rolled-up frame 10 and flap 15 with the longitudinal axis of hollow needle 35. In this form of the invention, finger anchors 20 of valve 5 may be similarly rolled up about the same single axis as frame 10 and flap 15 or, alternatively, they may extend longitudinally, e.g., parallel to the axis of rolled-up frame 10 and flap 15.

In use, and still looking at FIGS. 7-9, needle 35 is passed through the skin of the patient and then through blood vessel V at the desired location. Note that needle 35 extends transverse to blood vessel V at the desired location, and not parallel to the longitudinal axis of blood vessel V, since valve 5 is advanced transversely into blood vessel V and not endoluminally along blood vessel V. Needle 35 does not have to go through the skin of a patient if the procedure is performed laparoscopically. Note that in one preferred form of the invention, needle 35 passes through both the proximal and distal walls of blood vessel V.

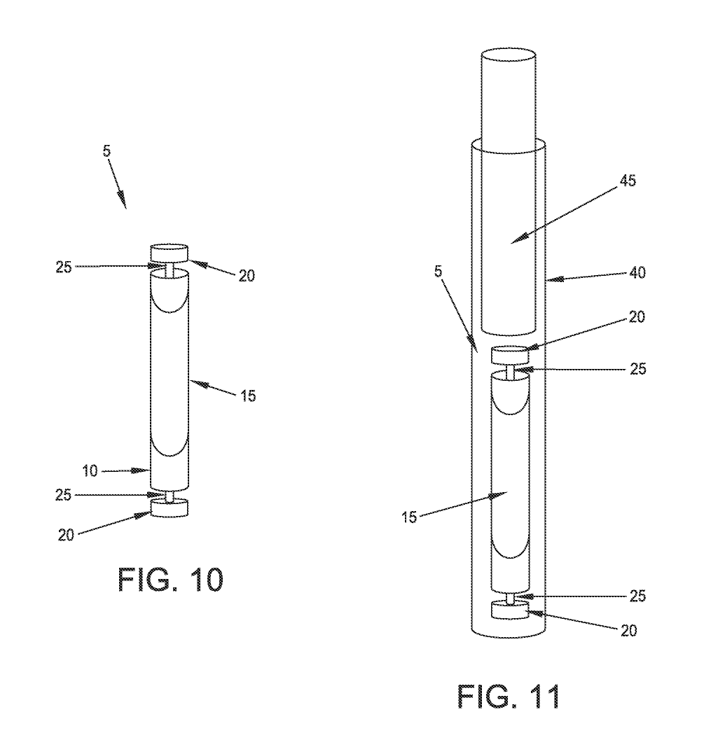

Looking now at FIGS. 10 and 11, there is shown a deployment element (or pusher) 45 that fits within sheath 40 and is used to push valve 5 through needle 35 while valve 5 is in its compressed state, as will hereinafter be discussed in greater detail.

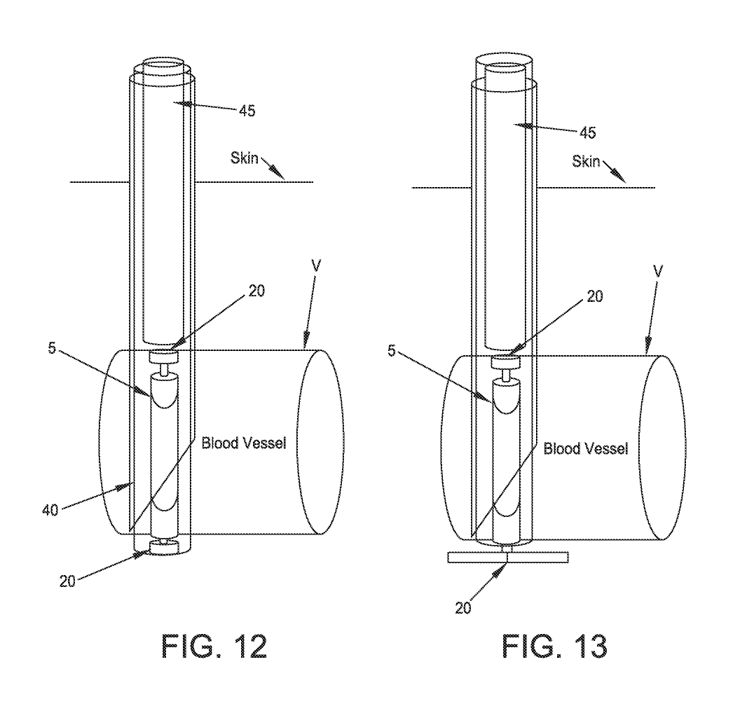

Looking next at FIGS. 7-9 and 12-15, needle 35 is advanced through the proximal wall of the blood vessel V, across the lumen of the blood vessel, and then through the distal wall of blood vessel V. Sheath 40 and valve 5 (in its compressed condition) are advanced together through the bore of needle 35 by maintaining pressure on the proximal end of valve 5 with pusher 45 while both sheath 40 and valve 5 are advanced distally through the bore of needle 35. Needle 35 is then retracted proximally, whereby to expose sheath 40 (FIG. 12). At this point, sheath 40 and finger anchors 20 of valve 5 protrude out of the distal wall of blood vessel V.

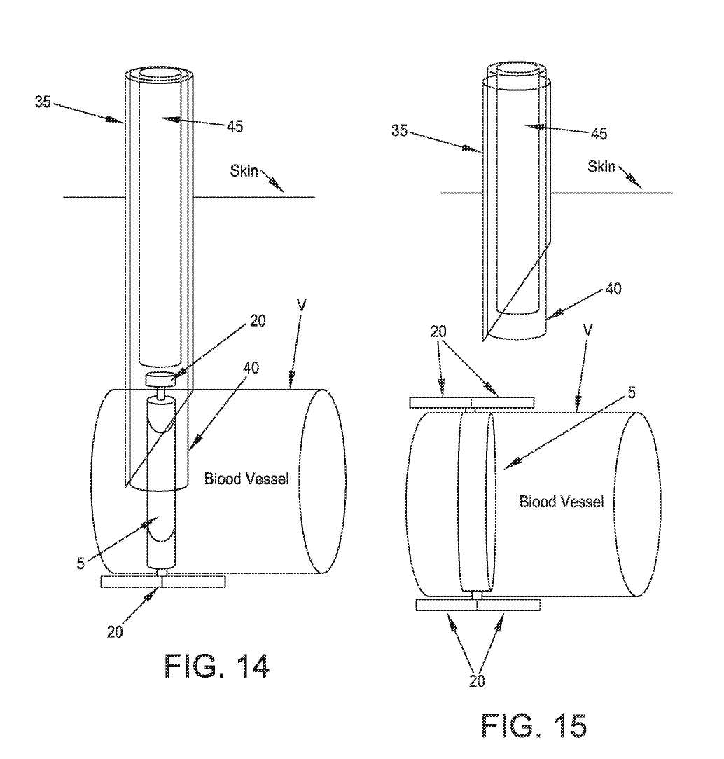

Sheath 40 and needle 35 are then retracted while valve 5 is maintained in position using pusher 45. See FIGS. 13 and 14. This causes resilient finger anchors 20 to open up on the distal side of blood vessel 20. Sheath 40 and needle 35 continue to be retracted until sheath 40 and needle 35 have been completely retracted from blood vessel V, thereby allowing resilient frame 10 and resilient flap 15 of valve 5 to “open up” and deploy within the lumen of the blood vessel V, anchored in place by resilient finger anchors 20 disposed external to blood vessel V (i.e., on both the distal and proximal sides of the blood vessel). See FIG. 15.

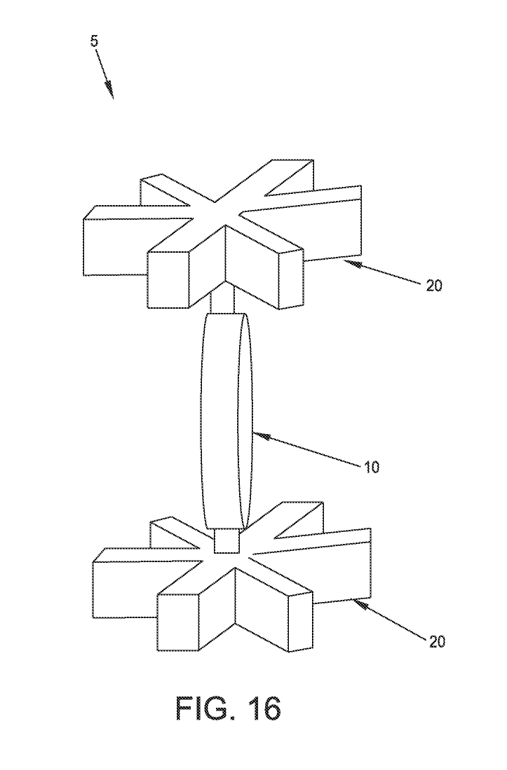

It should be appreciated that the number, configuration and geometry of finger anchors 20 may vary depending on the specific needs of the clinical application. By way of example but not limitation, FIG. 16 shows a valve 5 having six finger anchors 20, although fewer or more finger anchors 20 may be included if desired.

Furthermore, it should be appreciated that resilient finger anchors 20 may be replaced by any structure, such as a disk for example, that opens up outside a blood vessel, whereby to anchor valve 5 within a blood vessel and secure valve 5 to the wall of the blood vessel. By way of example but not limitation, finger anchors 20 may comprise a Nitinol or plastic or polymer ball, a coiled spring, etc. Alternative embodiments are possible and will be evident to those skilled in the art in view of the present disclosure.

In an alternative form of the present invention, and looking now at FIG. 17, an aperture 50 may be provided in flap 15 of valve 5. Aperture 50 can be selectively sized so as to enable a desired amount of blood to flow back-and-forth through valve 5. By way of example but not limitation, aperture 50 may be useful if a minimum amount of blood flow through the blood vessel is desired, while halting most of blood flow in a given direction. This permits a blood vessel to be perfused with flowing blood at all times, which may be advantageous in keeping a desired organ alive, or which may minimize the impact of a lack of blood flow in a particular direction in a blood vessel.

A valve 5 having an aperture 50 formed in flap 15 (FIG. 17) can be used to reduce the pressure of blood flow on the distal side of the valve, whereby to reduce blood pressure in the distal side of the valve, e.g., down the Saphenous vein if the valve is deployed next to the Sapheno-Femoral junction.

If desired, a plurality of valves 5 may be deployed in a single blood vessel, or a plurality of valves 5 may be deployed in multiple blood vessels, so as to physiologically control blood flow and pressure within a blood vessel in a minimally invasive manner, and without requiring the destruction of, or physical removal of, the blood vessels.

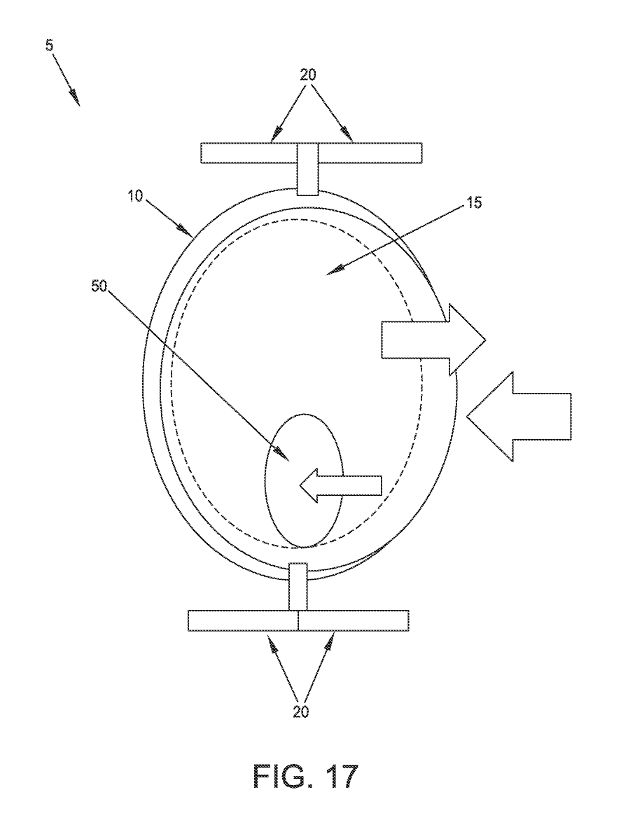

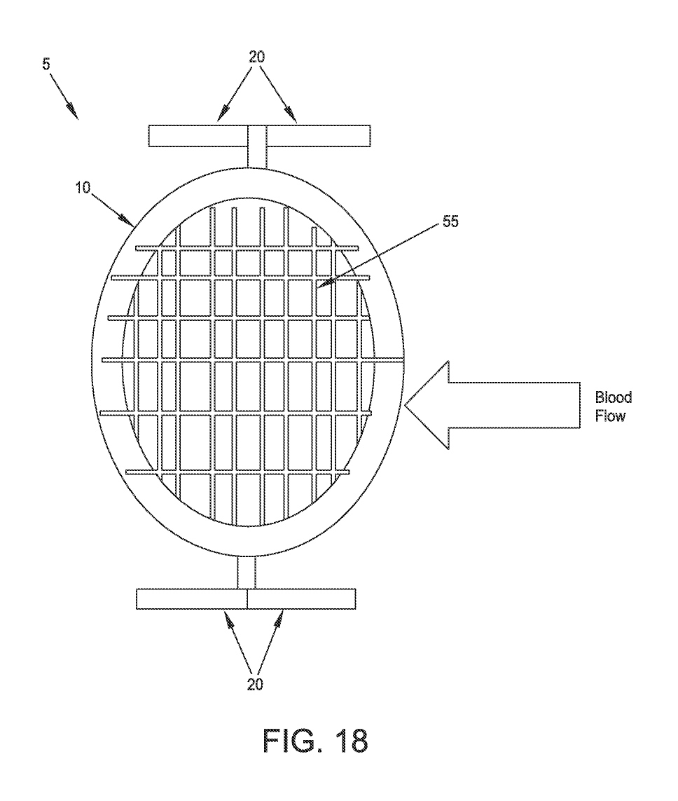

In yet another form of the present invention, and looking now at FIG. 18, flap 15 may comprise a filter 55 which can be deployed to “catch” (i.e., filter out) emboli and thereby prevent blood clots from getting to critical organs in the body (e.g., the heart, lungs, brain, etc.). In this form of the invention, filter 55 comprises a structure (e.g., a mesh, a permeable membrane, or any other filtering structure) which allows blood to flow through filter 55 but which will not permit larger structures (e.g., blood clots) to flow though filter 55. Filter 55 may comprise a polymer that dissolves over time, leaving just an open frame 10 in the blood vessel, or the entire structure (i.e., frame 10 and filter 55) could be made of a resorbable material and resorb over time.

It should be appreciated that filter 55 (or any other flow control device formed in accordance with the present invention) may be positioned at an angle relative to the blood flow, such that blood may flow across both sides of filter 55. By way of example but not limitation, the degree to which a vessel is occluded may be defined by the angle between the blood flow and the plane of filter 55, e.g., if the angle is 20 degrees, blood will flow through the blood vessel, even though an occlusion device (i.e., filter 55) may be deployed in the blood vessel. The angle of filter 55 (or other flow control device) relative to the flow of blood in the blood vessel may be adjusted externally to the blood vessel, or re-adjusted as needed over time, without having to penetrate the blood vessel.

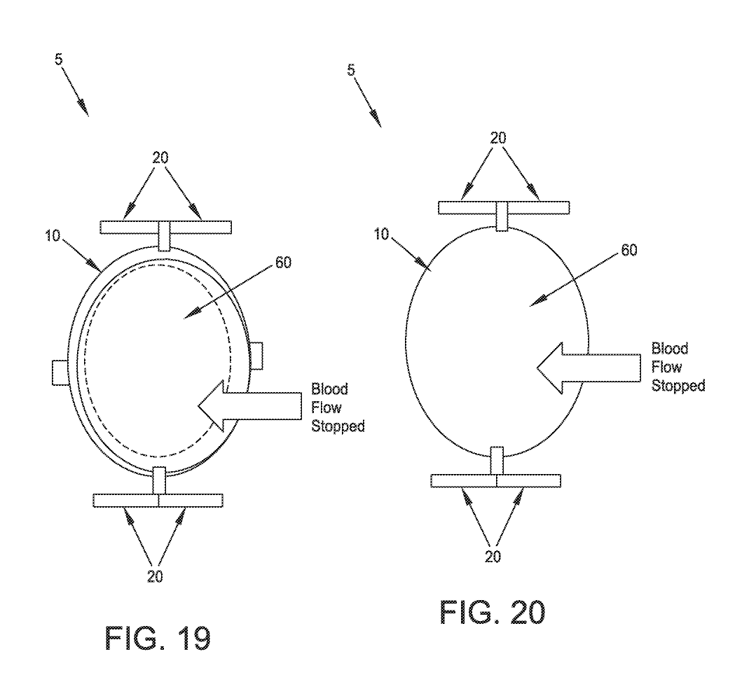

In still another embodiment of the present invention, and looking now at FIGS. 19 and 20, flap 15 may comprise a solid barrier 60 which prevents blood from flowing through frame 10, whereby to completely occlude the blood vessel. Barrier 60 may be formed out of the same material as frame 10, as a contiguous piece, or barrier 60 may comprise a material that is different in thickness, density, shape, etc. The angle of barrier 60, relative to the direction of blood flow, can be used as a means of defining and selecting the level of occlusion of the blood vessel, and allowed blood flow through the blood vessel.

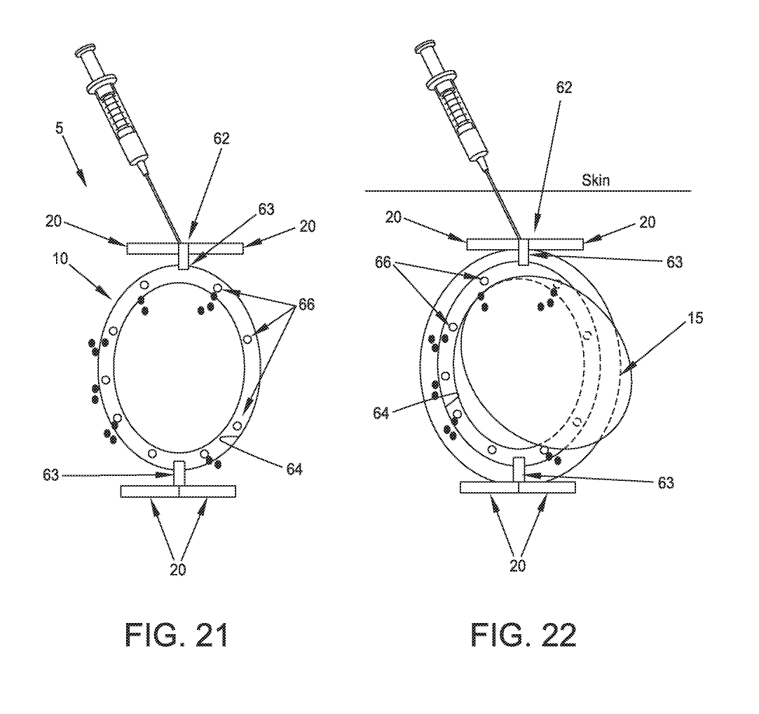

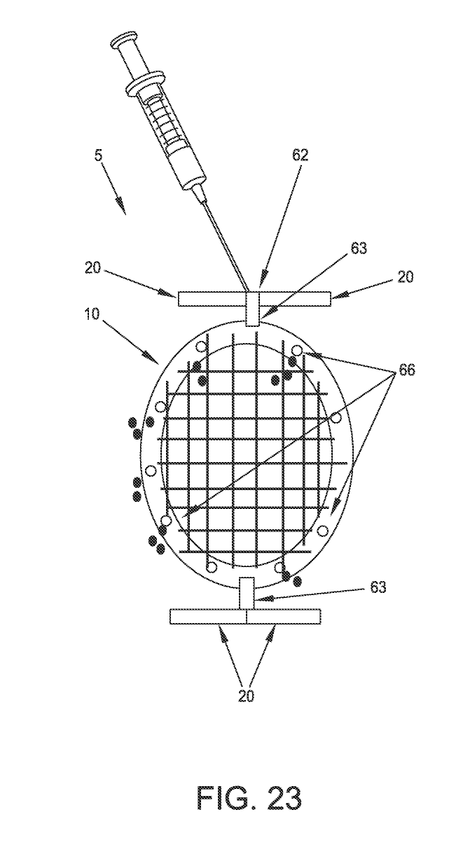

In still another embodiment of the present invention, and looking now at FIGS. 21-23, frame 10 may be formed hollow, or frame 10 may contain one or more channels for transporting a liquid (e.g., a thrombolytic agent, a drug, etc.) from an injection port (or reservoir) 62, though a conduit 63, and into channels 64 formed in frame 10, and then out of holes 66 formed in frame 10, whereby to deliver the solution into blood vessel V (or other tubular structure). Injection port 62 is formed such that it can receive a needle (or other tube) which is inserted through the skin, whereby fluid can be injected into the flow control device. By way of example but not limitation, holes 66 may be of different sizes, e.g., holes 66 may be formed smaller when closer to injection port 62, and holes 66 may be formed larger when further away from injection port 62, so as to ensure uniform distribution of the fluid being injected into injection port 62 and ejected out holes 66. If desired, a plurality of injection ports 62 may be provided (e.g., to supply a thrombolytic agent to both sides of the flow control device, and/or upstream and downstream of the flow control device, etc.).

Channels 64 formed in frame 10 communicate with the hollow tube or blood vessel via holes 66 such that the injected fluid enters into channels 64 and passes out of holes 66 of the porous openings into any attached clot, thereby promoting dissolution of this clot and allowing the full function of the valve (or other flow control device). This ensures a long operating life for the valve 5 (or flow control device), generally much longer than is typically achieved. Injection port (or reservoir) 62 may contain the fluid for a period of time, slowly dispensing the fluid over a period of time in a controlled release, thereby preventing buildup of thrombus, or delivering a desired substance to the tubular structure for a desired period of time. Multiple injection ports (or reservoirs) 62 may be connected to frame 10, enabling simplified access, or delivery of more fluid or fluids that may mix in channels 64 formed in frame 10.

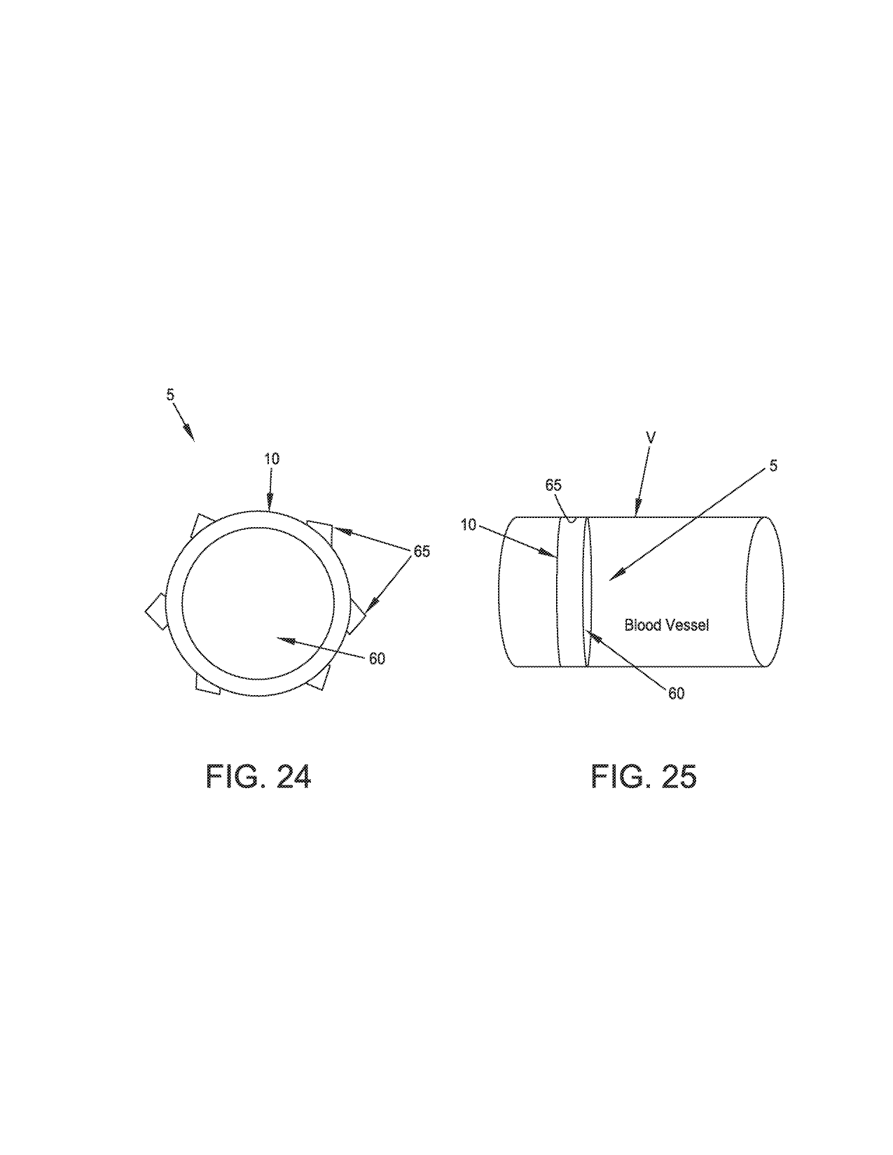

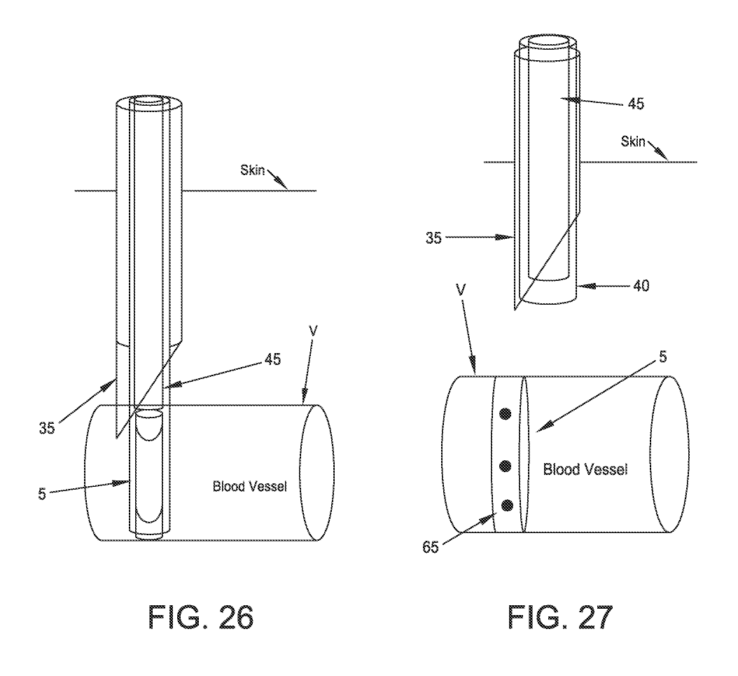

In yet another embodiment of the present invention, and looking now at FIGS. 24-27, valve 5 can be constructed so that it does not have finger anchors 20 residing outside of the blood vessel. In this form of the invention, frame 10 comprises spikes (or grips) 65 which allow frame 10 to grip the internal wall of the blood vessel, and/or to penetrate partially or fully through the wall of the blood vessel, whereby to anchor valve 5 in position. In this embodiment of the present invention, valve 5 is deployed within the interior of a blood vessel in a manner similar to that by which the embodiment of FIGS. 7-15 is deployed, i.e., using a needle 35, except that with this form of the invention, needle 35 (and/or sheath 40) need not penetrate the distal wall of blood vessel V, since valve 5 can be deployed entirely within the interior of blood vessel V. Thus, in this form of the invention, needle 35 only needs to penetrate the surface of the skin, intervening tissue lying between the surface of the skin and the blood vessel, and the proximal wall of the blood vessel.

However, it should also be appreciated that, if desired, both finger anchors 20 and spikes 65 may be provided on frame 10 in order to secure valve 5 within a blood vessel.

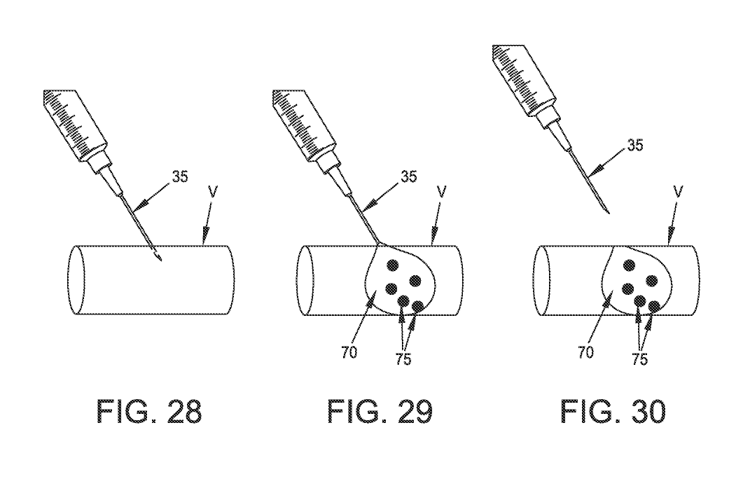

In still another embodiment of the present invention, and looking now at FIGS. 28-30, a balloon 70, having anchor elements 75 (e.g., barbs, protrusions, roughened surfaces, etc.) disposed on its outer surface, may be percutaneously delivered (e.g., via needle 35) into blood vessel V, and then balloon 70 may be inflated via needle 35, whereby to occlude blood vessel V. In this form of the invention, anchor elements 75 can enhance the adhesion of the balloon to the interior walls of blood vessel V (e.g., by a friction fit). Balloon 70 may be inflated with a fluid (e.g., air, saline, etc.), or balloon 70 may be filled with a solidifying polymer (or other materials) so that balloon 70 solidifies after it is expanded in place.

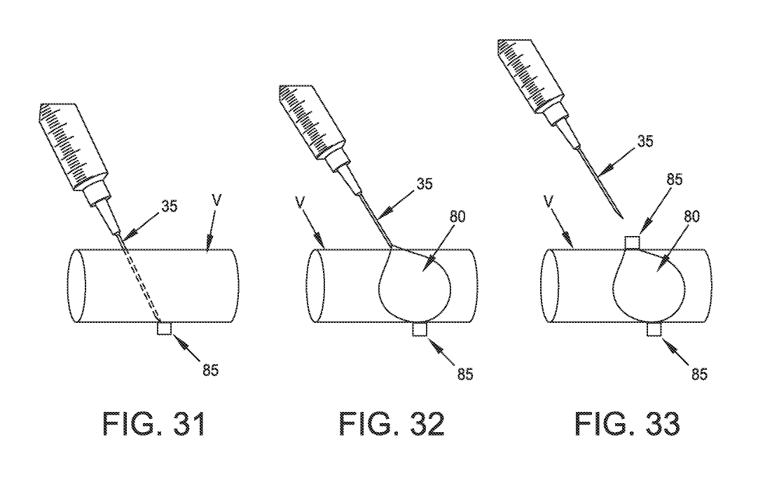

Alternatively, and looking now at FIGS. 31-33, balloon 70 may be replaced with an occlusion element 80 which comprises a polymer matrix material (or glue) such that it forms a solid occlusion of only a portion of, or the entirety of, blood vessel V. Occlusion element 80 is formed directly at the site where needle 35 penetrates blood vessel V, so multiple occlusion elements 80 can be formed in the same (or multiple) blood vessels V. The polymer matrix may comprise Super Glue, Crazy Glue, or any other satisfactory material. The polymer matrix which comprises occlusion element 80 may be resorbable, and/or the polymer matrix may be temperature sensitive. Occlusion element 80 may also penetrate blood vessel V in at least two locations.

As seen in FIGS. 31-33, if desired, occlusion element 80 may be anchored in position by polymer anchors 85, which solidify, harden and penetrate through the wall of blood vessel V, whereby to fill the void created by needle 35 as it punctures blood vessel V. If desired, balloon 70 can be fabricated with protrusions which correspond to polymer anchors 85, such that the balloon protrusions extend through the side wall of the blood vessel, whereby to anchor the balloon to the blood vessel.

It should be appreciated that the flow control elements described above (i.e., valve 5, frame 10, flap 15, filter 55, barrier 60, balloon 70, occlusion element 80 and polymer anchors 85), and the anchoring elements discussed above (i.e., finger anchors 20, connector 25, anchors 30, spikes 65 and anchor elements 75) may comprise, but are not limited to, the following: biocompatible metals (e.g., Nitinol, Titanium, etc.) or various polymers that may be hard, soft, and/or flexible, and which may be permanent or absorbable or bioresorbable. Examples of such polymers include, but are not limited to, PGA, PLA (Poly Lactic Acid), PCL, PLGA, PLC, PLLA, poly-lactide, Poly Hydroxy Alkanoates, polymer alkylene bis(dilactoyl)-methacrylate, Block Co-Polymers, or Silk derivatives. The surrounding structure may be made of a hard polymer (which may be more crystalline if desired), shape memory metals, or polymers. Additionally, the present invention may be formed out of Super Glue, Crazy Glue, CyanoAcrylate, ceramics, carbide materials, etc.

The injectable flow control devices may be made of bio-compatible metals in combination with a polymer-polymer, or a polymer mixed with other compounds to optimize mechanical, inertness and other characteristics.

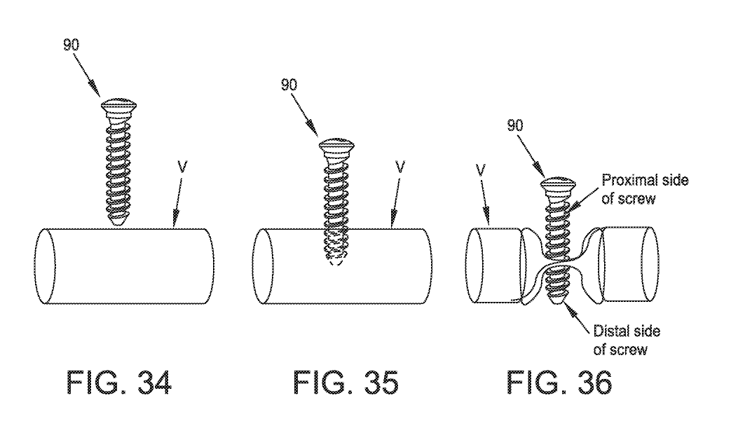

In another form of the present invention, and looking now at FIGS. 34-36, a transvascular screw 90 may be used to occlude blood vessel V. Transvascular screw 90 may comprise an implantable biocompatible resorbable or non-resorbable polymer, or plastic, or silk, or a hard metal or other material. In use, transvascular screw 90 (or multiple transvascular screws 90) is screwed across blood vessel V so as to pull the proximal wall and the distal wall of the blood vessel V together, whereby to occlude blood vessel V. Transvascular screw 90 may also comprise a second transvascular screw (or mechanism) disposed within the interior of transvascular screw 90, such that the distal and proximal ends of transvascular screw 90 can be selectively expanded by actuating the second transvascular screw (or mechanism) whereby to help lock transvascular screw 90 to the side wall of blood vessel V. The pitch of the threads disposed on the outer surface of transvascular screw 90, or the diameter of the threads of transvascular screw 90, may also be variable, whereby to control occlusion strength or other characteristics of the occlusion.

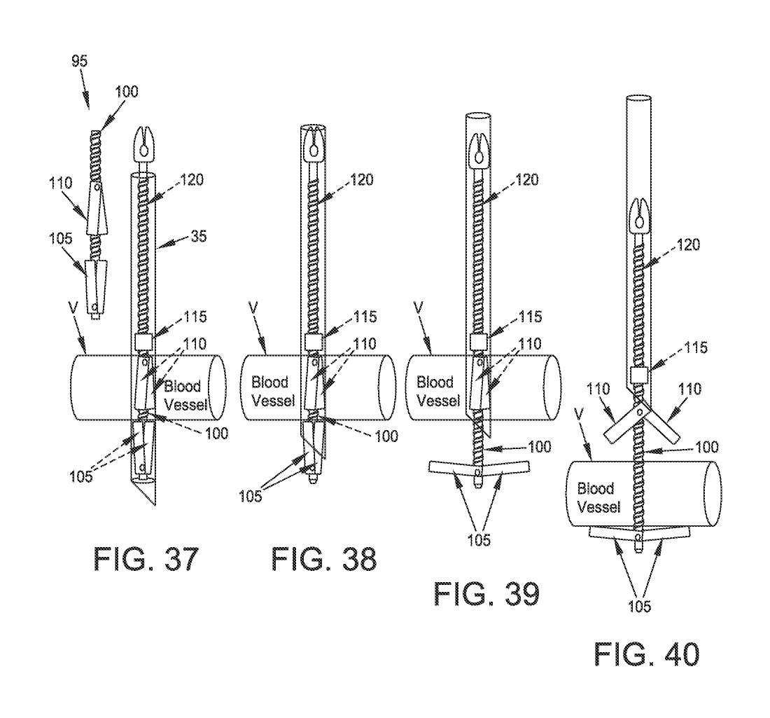

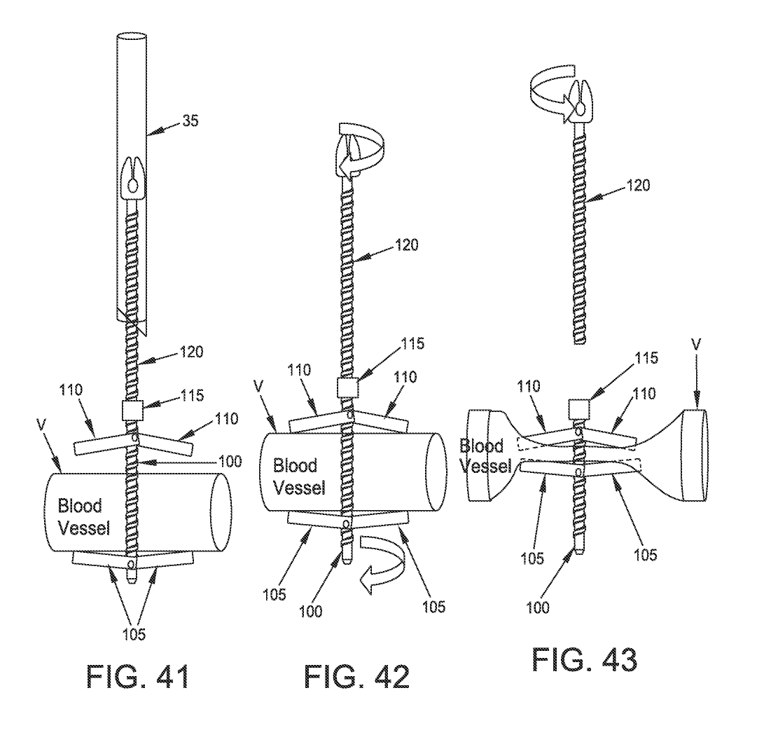

In still another form of the present invention, and looking now at FIGS. 37-43, a transvascular clamp 95 is provided for effecting occlusion of a blood vessel V.

Transvascular clamp 95 comprises a shaft 100 having a plurality of distal fingers 105 and a plurality of proximal fingers 110. In one preferred form of the invention, shaft 100 is threaded and fingers 105, 110 are threadingly engaged with shaft 100, such that rotation of shaft 100 in a direction causes fingers 105, 110 to pivot outward. Shaft 100 terminates in a coupling 115 disposed at the proximal end of shaft 100. Coupling 115 comprises a threaded bore, whereby to releasably attach a rod 120 to coupling 115. When rod 120 is rotated in a first direction, shaft 100 advances distally. When rod 120 is rotated in a second, opposite direction, rod 120 disengages from coupling 115. Fingers 105 can be folded against shaft 100 of transvascular clamp 95 so as to assume a compact configuration for percutaneous delivery (e.g., via a needle 35) to a blood vessel V. In use, transvascular clamp 95 is disposed in sheath 40 with fingers 105, 110 folded against shaft 100 of transvascular clamp 95 (i.e., transvascular clamp 95 is in its compact configuration), and sheath 40 is disposed within the bore of hollow needle 35. Hollow needle 35, sheath 40 and transvascular clamp 95 are advanced through the skin of the patient and through the proximal and distal walls of blood vessel V, whereby to span the lumen of blood vessel V with shaft 100 of transvascular clamp 95. Next, sheath 40 and needle 35 are retracted proximally. When needle 35 and sheath 40 are removed by retracting proximally, the distal fingers 105 of transvascular clamp 95 open up, either independently (e.g., via a spring action) or by rotating shaft 100. When the needle is retracted to expose the proximal fingers 110, proximal fingers 110 of occlusion device 95 open up, either independently (e.g., via a spring action) or by rotating shaft 100. Once the fingers 105, 110 are exposed, shaft 100 is rotated further so that the fingers 105, 110 are brought together across blood vessel V, whereby to occlude blood vessel V. Rod 120 is then rotated in the opposite direction to disengage rod 120 from coupling 115, leaving the implanted transvascular clamp 95 disposed across the blood vessel V.

Shaft 100 may be made of metal or polymers that may absorb or remain permanently. This and other occlusion devices described above may be used in conjunction with sclerosants, glues, laser and RF ablation probes to protect the deep vein system as well as protect the patient from embolization threats. The occlusion device may be used to occlude the vein at a site of interest and the glues or other elements may be used to occlude an entire portion of a blood vessel.

### Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for controlling flow through a body lumen, the apparatus comprising:
an injectable flow control device for disposition within the body lumen, the injectable flow control device comprising:
a resilient frame for seating against the inside wall of the body lumen and compressible for disposition within a tube for delivery through the side wall of the body lumen to the interior of the body lumen, the resilient frame having an opening therein; and
a flow restrictor in contact with the resilient frame for restricting flow through the opening of the resilient frame.

2. Apparatus according to claim 1 wherein the tube comprises a needle.

3. Apparatus according to claim 2 wherein the flow restrictor is formed integral with the resilient frame.

4. Apparatus according to claim 2 wherein the resilient frame comprises at least one anchor for securing the resilient frame to the side wall of the body lumen.

5. Apparatus according to claim 4 wherein at least one anchor extends through the side wall of the body lumen.

6. Apparatus according to claim 5 wherein at least one anchor comprises at least one laterally-extending element for disposition outside of the body lumen.

7. Apparatus according to claim 5 wherein at least two anchors extend through diametrically-opposed portions of the side wall of the body lumen.

8. Apparatus according to claim 4 wherein at least two anchors extend into the side wall of the body lumen.

9. Apparatus according to claim 4 wherein the at least two anchors comprise barbs which extend along the perimeter of the resilient frame.

10. Apparatus according to claim 2 wherein the injectable flow control device comprises a valve.

11. Apparatus according to claim 10 wherein the flow restrictor is movably mounted to the resilient frame so as to selectively (i) permit flow in one direction through the opening of the resilient frame, and (ii) prevent flow in the opposing direction through the opening of the resilient frame.

12. Apparatus according to claim 2 wherein the injectable flow device comprises a filter.

13. Apparatus according to claim 12 wherein the flow restrictor comprises a mesh.

14. Apparatus according to claim 2 wherein the injectable flow device comprises an occluder.

15. Apparatus according to claim 14 wherein the flow restrictor comprises a barrier formed out of a fluid-impermeable material.

16. Apparatus according to claim 15 wherein the barrier has an opening formed therein.

17. Apparatus according to claim 2 further comprising a needle which receives the injectable flow control device.

18. Apparatus according to claim 17 further comprising a sheath which receives the injectable flow control device, wherein the sheath is received by the needle.

19. Apparatus according to claim 2 wherein the injectable flow control device comprises delivery means for delivering an agent to the region about the injectable flow control device.

20. Apparatus according to claim 19 wherein the delivery means comprise at least one channel in the resilient frame.

21. Apparatus according to claim 20 wherein the agent comprises a thrombolytic agent for reducing clotting.

22. Apparatus according to claim 20 where the delivery means may be accessed without requiring penetration of the body lumen.

23. Apparatus for controlling flow through a body lumen, the apparatus comprising:
an injectable flow control device for disposition within the body lumen, the injectable flow control device comprising:
a balloon for seating against the inside wall of the body lumen and compressible for disposition within a tube for delivery through the side wall of the body lumen to the interior of the body lumen.

24. Apparatus according to claim 23 wherein the tube comprises a needle.

25. Apparatus according to claim 24 wherein the balloon comprises a plurality of anchor elements disposed on the outer surface of the balloon for engaging the side wall of the body lumen.

26. Apparatus according to claim 24 wherein at least one portion of the balloon extends through the side wall of the body lumen, whereby to secure the balloon in the body lumen.

27. Apparatus for controlling flow through a body lumen, the apparatus comprising:
an injectable flow control device for disposition within the body lumen, the injectable flow control device comprising:
a flowable material for solidifying against the inside wall of the body lumen, wherein at least one portion of the solidified material extends through the side wall of the body lumen, whereby to secure the solidified material in the body lumen.

28. A method for controlling flow through a body lumen, the method comprising:
advancing a tube through the side wall of a body lumen; and
positioning an injectable flow control device within the body lumen, the injectable flow control device controlling flow through a body lumen.

29. A method according to claim 28 wherein the tube comprises a needle.

30. A method according to claim 29 wherein the injectable flow control device is contained within the needle as the needle is advanced through the side wall of the body lumen.

31. A method according to claim 30 wherein the injectable flow control device is disposed in a rolled-up configuration when it is contained within the needle.

32. A method according to claim 29 wherein the needle is advanced through a proximal side wall of the body lumen, across the body lumen, and through the distal side wall of the body lumen.

33. A method according to claim 32 wherein a first portion of the injectable flow control device is deployed from the needle beyond the distal side wall of the body lumen, a second portion of the injectable flow control device is deployed from the needle in the body lumen, and a third portion of the injectable flow control device is deployed from the needle on the near side of the proximal side wall of the body lumen.

34. A method according to claim 29 wherein the injectable flow control device comprises:
a resilient frame for seating against the inside wall of the body lumen and compressible for disposition within the needle, the resilient frame having an opening therein; and
a flow restrictor mounted to the resilient frame for restricting flow through the opening of the resilient frame.

35. A method according to claim 34 wherein the injectable flow control device comprises a valve.

36. A method according to claim 35 wherein the flow restrictor is movably mounted to the resilient frame so as to selectively (i) permit flow in one direction through the opening of the resilient frame, and (ii) prevent flow in the opposing direction through the opening of the resilient frame.

37. A method according to claim 34 wherein the injectable flow device comprises a filter.

38. A method according to claim 37 wherein the flow restrictor comprises a mesh.

39. A method according to claim 34 wherein the injectable flow device comprises an occluder.

40. A method according to claim 39 wherein the flow restrictor comprises a barrier formed out of a fluid-impermeable material.

41. A method according to claim 40 wherein the barrier has an opening formed therein.

42. A method according to claim 28 further comprising periodically delivering an agent which permeates through the injectable flow control device to reduce thrombosis.
